SEQUENCE LISTING (7 sequences)

SEQ ID NO: 1 - protein, 15 aa, synthetic construct
  SITE 1..15: note = AviTag
GLNDIFEAQKIEWHE

SEQ ID NO: 2 - protein, 26 aa, synthetic construct
  SITE 1..26: note = Calmodulin-tag
KRRWKKNFIAVSAANRFKKISSSGAL

SEQ ID NO: 3 - protein, 15 aa, synthetic construct
  SITE 1..15: note = S-tag
KETAAAKFERQHMDS

SEQ ID NO: 4 - protein, 38 aa, synthetic construct
  SITE 1..38: note = SBP-tag
MDEKTTGWRGGHVVEGLAGELEQLRARLEHHPQGQREP

SEQ ID NO: 5 - protein, 8 aa, synthetic construct
  SITE 1..8: note = Strep-tag II
WSHPQFEK

SEQ ID NO: 6 - protein, 16 aa, synthetic construct
  SITE 1..16: note = Isopeptag
TDKDMTITFTNKKDAE

SEQ ID NO: 7 - protein, 13 aa, synthetic construct
  SITE 1..13: note = Spytag
AHIVMVDAYKPTK

(19) United States
(12) Patent Application Publication
(10) Pub. No.: US 2026/0069682 A1
(43) Pub. Date: Mar. 12, 2026

(54) NANOPARTICLE-DERIVED VACCINES AGAINST POXVIRUSES, AND METHODS FOR MAKING AND USING THE SAME

(71) Applicant: The United States of America as Represented By The Secretary of the Department of Health and Human S, Bethesda, MD (US)

(72) Inventors: Bernard MOSS, Bethesda, MD (US); Huibin YU, Bethesda, MD (US); Wolfgang RESCH, Kensington, MD (US); Ahmed BELGHITH, Washington, DC (US); Robert Pearce HYATT, Portland, OR (US)

(73) Assignee: The United States of America as Represented By The Secretary of the Department of Health and Human S, Bethesda, MD (US)

(21) Appl. No.: 19/107,331

(22) PCT Filed: Aug. 31, 2023
(86) PCT No.: PCT/US2023/031683

§ 371 Date: Feb. 27, 2025

Related U.S. Application Data
(60) Provisional application No. 63/402,702, filed on Aug. 31, 2022.

(51) Int. Cl. A61K 39/285; A61K 39/00; A61K 39/275; A61P 37/04

(52) U.S. Cl. CPC A61K 39/285; A61K 39/275; A61P 37/04; A61K 2039/5258; A61K 2039/55555

(57) ABSTRACT

The present disclosure relates generally to vaccines against orthopoxviruses, and methods for making and using such vaccines. In particular, in some embodiments, the present disclosure relates to nanoparticle-derived vaccines, and compositions based thereon, that elicit an immune response against an orthopoxvirus. The present disclosure further relates to the use of vaccines and vaccine compositions for preventing; decreasing the severity, morbidity and/or mortality of; shortening the duration of; and/or reducing the symptoms of, a poxvirus infection, such as, for example, an orthopoxvirus infection.

### CROSS REFERENCE TO RELATED APPLICATIONS

This application claims the benefit of and the priority to U.S. Provisional Application No. 63/402,702 filed Aug. 31, 2022, of which the entire content is incorporated by reference in its entirety.

### STATEMENT REGARDING FEDERALLY SPONSORED RESEARCH OR DEVELOPMENT

This invention was made with government support under National Institute of Allergy and Infectious Diseases (NIAID) intramural project number AI000416-39, awarded by the Department of Health and Human Services (DHHS). The government has certain rights in the invention.

### INCORPORATION BY REFERENCE OF SEQUENCE LISTING

The content of the electronically submitted sequence listing, file name: F289607 SEQ_LIS_ST26_AS FILED.xml; size: 9,859 bytes; and date of creation: Aug. 13, 2023, filed herewith, is incorporated herein by reference in its entirety.

### FIELD

The present disclosure relates generally to vaccines against poxviruses, and methods for making and using such vaccines. In particular, in some embodiments, the present disclosure relates to nanoparticle-derived vaccines, and compositions based thereon, that elicit an immune response against orthopoxviruses. The present disclosure further relates to the use of vaccines and vaccine compositions for preventing; decreasing the severity, morbidity and/or mortality of; shortening the duration of; and/or reducing the symptoms of, a poxvirus infection, such as, for example, a monkeypox virus infection.

### BACKGROUND

Two orthopoxviruses including smallpox virus and mpox (monkeypox) virus cause severe disease. Mpox was historically a zoonosis restricted to Central and West Africa where it is endemic. Recently, however, it has been reported that mpox spread globally.

The current monkeypox virus outbreak, which was initially reported in the United Kingdom in May 2022, has caused more than 41,000 reported cases (and at least 12 deaths) as of August 2022. This outbreak differs from previous monkeypox virus outbreaks not just in the vastly increased number of cases, but also in its global distribution; the overwhelming majority of the 41,000 cases (40,971) have been reported in locations that have not historically reported monkeypox infection. See, for example, www.cdc.gov/poxvirus/monkeypox/response/2022/world-map.html. The number steadily increased to more than 86,000 cases in over 120 countries as of July 2023.

Although immunization with existing smallpox vaccines (using live, replicating- or non-replicating, vaccinia virus) appears to provide some protection against monkeypox virus infection (based on data from previous monkeypox outbreaks, see www.cdc.gov/poxvirus/monkeypox/clinicians/smallpox-vaccine.html), it is noteworthy that routine vaccination against smallpox ended about fifty years ago (in 1972 in the United States, for example). Therefore, younger generations have never received the vaccine; and immunity to smallpox may now be waning in older vaccinated generations. There is thus a need for improved vaccines against monkeypox.

Current (Jynneos) mpox vaccine is difficult to manufacture in large quantities and does not completely prevent infection, indicating a need for a new vaccine.

The inventors found through animal studies that soluble L1 protein of the vaccinia virus mature virion (MV) membrane and A33 and B5 proteins of the extracellular enveloped virion (EV) membrane protect against vaccinia, and further noted mpox virus challenges due to their high conservation.

Therefore, there still is a need for improved mpox vaccines with enhanced neutralizing responses.

### SUMMARY OF THE INVENTION

The present disclosure addresses the above-described limitations in the art, by providing, amongst others, vaccines and vaccine compositions for preventing; decreasing the severity, morbidity and/or mortality of, shortening the duration of; and/or reducing the symptoms of, a poxvirus infection, such as, for example, a monkeypox virus infection. The present disclosure further relates to nanoparticle-derived vaccines, and compositions based thereon, that elicit an immune response against at least one poxvirus of interest, such as a monkeypox virus.

Non-limiting embodiments of the disclosure include as follows:

[1] A vaccine composition for eliciting an immune response against a poxvirus, wherein said vaccine composition comprises nanoparticles with at least one poxvirus antigen attached thereto.

[2] The composition of [1], wherein said at least one poxvirus antigen is a protein, or fragment, variant, or derivative thereof, from a poxvirus selected from the group consisting of vaccinia virus, monkeypox virus, and variola virus.

[3] The composition of [1] or [2], wherein a plurality of different poxvirus antigens is attached to said nanoparticles.

[4] The composition of [3], wherein the plurality of different poxvirus antigens comprises poxvirus antigens from different poxviruses.

[5] The composition of [3], wherein the different poxvirus antigens are from the same poxvirus.

[6] The composition of [5], wherein the different poxvirus antigens are from monkeypox virus, vaccinia virus, or combinations thereof.

[7] The composition of [1]-[6], wherein at least three different poxvirus antigens are attached to said nanoparticles.

[8] The composition of [7], wherein said at least three different poxvirus antigens are proteins, or fragments, variants, or derivatives thereof, of a poxvirus protein selected from L1, A33, B5, A28, H2, A16, and G9 from vaccinia virus, and their respective homologs M1, A35, B6, A30, H2, A17, and G10 from monkeypox virus.

[9] The composition of [7], wherein the at least three different poxvirus antigens comprise L1, A33, and B5 proteins from one or more poxviruses, or fragments, variants, or derivatives thereof.

[10] The composition of [7], wherein the at least three different poxvirus antigens comprise L1, A33, and B5 proteins from vaccinia virus and/or their respective homologs M1, A35 and B6 proteins from monkeypox virus, or fragments, variants, or derivatives thereof.

[11] The composition of [7], wherein the at least three different poxvirus antigens comprise L1, A33, B5, A28, and H2 proteins from vaccinia virus and/or their respective homologs M1, A35, B6, A30, and H2 proteins from monkeypox virus, or fragments, variants, or derivatives thereof.

[12] The composition of [1]-[11], wherein said at least one poxvirus antigen is fused to a peptide tag sequence that facilitates binding to said nanoparticles.

[13] The composition of [12], wherein said peptide tag sequence is a SpyTag sequence.

[14] The composition of [13], wherein said nanoparticles comprise a SpyCatcher polypeptide, and wherein said tag sequence binds to said SpyCatcher polypeptide.

[15] The composition of [14], wherein said SpyCatcher polypeptide is fused to a component, monomer, or peptide, of said nanoparticle.

[16] The composition of any one of [1]-[15], wherein said nanoparticles with at least one poxvirus antigen attached thereto are a virus-like particle (VLP).

[17] The composition of [1]-[16], wherein at least four different poxvirus antigens are attached to said nanoparticles.

[18] The composition of [1]-[17], wherein at least five different poxvirus antigens are attached to said nanoparticles.

[19] The composition of [1]-[18], wherein at least six different poxvirus antigens are attached to said nanoparticles.

[20] The composition of any one of [1], [3], [7] and [17]-[19], wherein said different poxvirus antigens are proteins, or fragments, variants, or derivatives thereof, of a poxvirus protein selected from vaccinia virus A26, A27, D8, H3, A16, A21, A28, F9, G3, G9, H2, J5, L1, L5, O3, A2.5, E10, G4, A9, A13, A14, A17, I2, A14.5, F14.5, I5, A27, A28, H3, D8, A13, A17, A33, and B5, or their respective monkeypox virus homologs.

[21] A method for eliciting an immune response against an orthopoxvirus in a subject, comprising administering an effective amount of the composition of any one of [1]-[20] to a subject.

[22] A method for immunizing against an orthopoxvirus, comprising administering an effective amount of the composition of any one of [1]-[20] to a subject.

[23] The method of any one of [21] and [22], wherein said administering is by intramuscular injection.

[24] The method of any one of [21] and [22], wherein the administration of said composition prevents, decreases the severity of, decreases the morbidity of, decreases the mortality of, shortens the duration of, and/or reduces the symptoms of, an orthopoxvirus infection.

### BRIEF DESCRIPTION OF DRAWINGS

FIG. 1 is schematic illustration of modifications of the genes encoding mpox proteins for attaching to nanoparticles, according to an embodiment.

FIG. 2A and FIG. 2B show the isolation of secreted M1 SpyTag and A35 SpyTag obtained in Example 1 according to an embodiment.

FIG. 3A schematically illustrates the attachment of mpox proteins to the nanoparticle according to an embodiment, and FIG. 3B shows SDS-PAGE results of A35 SpyTag and M1 SpyTag proteins attached to the nanoparticles in Example 2 according to an embodiment.

FIG. 4 is an illustration of vaccination timeline employed in Example 3 according to an embodiment.

FIG. 5 shows IgG endpoint titers measured at pre-bleed, first dose (prime), second dose (boost) and third dose (second boost) for the blood sample taken from mice administered with 2 μg of M1-nanoparticle complex (M1 2 μg) or A35-nanoparticle complex (A35 2 μg), 5 μg of M1-nanoparticle complex (M1 5 μg) or A35-nanoparticle complex (A35 5 μg), a mixture of 2 μg M1-nanoparticle complex and 2 μg A35-nanoparticle complex (M1/A35 4 μg), a mixture of 5 μg M1-nanoparticle complex and 5 μg A35-nanoparticle complex (M1/A35 10 μg), 10⁷ plaque forming units of modified vaccinia Ankara (MVA) or 5 μg of SpyCatcher003-mi3 (SC003-mi3).

FIG. 6 shows vaccinia virus Western Reserve strain (VACV-WR)-neutralizing activity (IC₅₀) measured at pre-bleed, first dose (prime), second dose (boost) and third dose (second boost) for the blood sample taken from mice administered with 2 μg of M1-nanoparticle complex (M1 2 μg) or A35-nanoparticle complex (A35 2 μg), 5 μg of M1-nanoparticle complex (M1 5 μg) or A35-nanoparticle complex (A35 5 μg), a mixture of 2 μg M1-nanoparticle complex and 2 μg A35-nanoparticle complex (M1/A35 4 μg), a mixture of 5 μg M1-nanoparticle complex and 5 μg A35-nanoparticle complex (M1/A35 10 μg), 10⁷ plaque forming units of MVA or 5 μg SpyCatcher003-mi3 (SC003-mi3).

FIG. 7 shows the body weight change rate of mice during 15 days after the VACV-WR challenge (infection).

FIG. 8 is a schematic diagram of H2-A28 fusion protein (wherein H2 and A28 proteins are truncated) according to an embodiment.

FIG. 9 is a vaccination timeline used in Example 6 according to an embodiment.

FIG. 10 and FIG. 11 show vaccinia virus Western Reserve strain (VACV-WR)-neutralizing activity (IC₅₀) and protective effects against VACV infection, respectively, by an empty vector plasmid, a plasmid encoding an H2-A28 fusion protein, individual plasmids encoding H2 and A28 proteins, by a single plasmid encoding both H2 and A28 proteins, by a soluble H2-A28 fusion protein, or adjuvant.

FIG. 12 is a schematic diagram of a vector for expressing soluble truncated vaccinia virus A16 and G9 as a heterodimer.

FIG. 13 and FIG. 14 vaccinia virus Western Reserve strain (VACV-WR)-neutralizing activity (IC₅₀) and protective effects against VACV infection, respectively, by A16/G9 heterodimer before immunization (PB) or after second and third immunization or after control injections of phosphate buffered saline (PBS). Percent of weight loss following challenge with vaccinia virus WR shown for two control mice (RP and NP) and mean values for all mice immunized with A16/G9

### INCORPORATION BY REFERENCE

All patents, publications, and patent applications cited in the present specification are herein incorporated by reference as if each individual patent, publication, or patent application was specifically and individually indicated to be incorporated by reference in its entirety for all purposes.

### DETAILED DESCRIPTION

As discussed herein, the present disclosure provides, amongst others, vaccines and vaccine compositions for preventing; decreasing the severity, morbidity and/or mortality of; shortening the duration of; and/or reducing the symptoms of, a poxvirus infection, such as, for example, an orthopoxvirus infection. The present disclosure further provides nanoparticle-derived vaccines, and compositions based thereon, that elicit an immune response against at least one poxvirus of interest, such as an orthopoxvirus.

It is to be understood that the terminology used herein is for the purpose of describing particular embodiments only and is not intended to be limiting. As used in the present specification and the appended claims, the singular forms “a,” “an,” and “the” include plural referents unless the context clearly dictates otherwise. Thus, for example, reference to “a polynucleotide” includes one or more polynucleotides, and reference to “a vector” includes one or more vectors.

Unless defined otherwise, all technical and scientific terms used herein have the same meaning as commonly understood by one of ordinary skill in the art to which the invention pertains. Although other methods and materials similar, or equivalent, to those described herein can be useful in the present invention, preferred materials and methods are described herein.

In view of the teachings of the present specification, one of ordinary skill in the art can apply conventional techniques of immunology, biochemistry, chemistry, molecular biology, microbiology, cell biology, genomics, and recombinant polynucleotides, as taught, for example, by the following standard texts: Abbas et al. (Cellular and Molecular Immunology, 2017, 9th Edition, Elsevier, ISBN 978-0323479783); Butterfield et al. (Cancer Immunotherapy Principles and Practice, 2017, 1st Edition, Demos Medical, ISBN 978-1620700976); Kenneth Murphy (Janeway's Immunobiology, 2016, 9th Edition, Garland Science, ISBN 978-0815345053); Stevens et al. (Clinical Immunology and Serology: A Laboratory Perspective, 2016, 4th Edition, Davis Company, ISBN 978-0803644663); E. A. Greenfield (Antibodies: A Laboratory Manual, 2014, Second edition, Cold Spring Harbor Laboratory Press, ISBN 978-1-936113-81-1); R. I. Freshney (Culture of Animal Cells: A Manual of Basic Technique and Specialized Applications, 2016, 7th Edition, Wiley-Blackwell, ISBN 978-1118873656); C. A. Pinkert (Transgenic Animal Technology, Third Edition: A Laboratory Handbook, 2014, Elsevier, ISBN 978-0124104907); H. Hedrich (The Laboratory Mouse, 2012, Second Edition, Academic Press, ISBN 978-0123820082); Behringer et al. (Manipulating the Mouse Embryo: A Laboratory Manual, 2013, Fourth Edition, Cold Spring Harbor Laboratory Press, ISBN 978-1936113019); McPherson et al. (PCR 2: A Practical Approach, 1995, IRL Press, ISBN 978-0199634248); J. M. Walker (Methods in Molecular Biology (Series), Humana Press, ISSN 1064-3745); Rio et al. (RNA: A Laboratory Manual, 2010, Cold Spring Harbor Laboratory Press, ISBN 978-0879698911); Methods in Enzymology (Series), Academic Press; Green et al. (Molecular Cloning: A Laboratory Manual, 2012, Fourth Edition, Cold Spring Harbor Laboratory Press, ISBN 978-1605500560); and G. T. Hermanson (Bioconjugate Techniques, 2013, Third Edition, Academic Press, ISBN 978-0123822390).

### Definitions

The terms “wild-type,” “naturally occurring,” and “unmodified” are used herein to mean the typical (or most common) form, appearance, phenotype, or strain existing in nature; for example, the typical form of viruses, cells, organisms, polynucleotides, proteins, macromolecular complexes, genes, RNAs, DNAs, or genomes as they occur in, and can be isolated from, a source in nature. The wild-type form, appearance, phenotype, or strain serve as the original parent before an intentional modification. Thus, mutant, variant, engineered, recombinant, and modified forms are not wild-type forms.

By “isolated” is meant, when referring to a polypeptide, that the indicated molecule is separate and discrete from the whole organism with which the molecule is found in nature or is present in the substantial absence of other biological macromolecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “purified” as used herein preferably means at least 75% by weight, more preferably at least 85% by weight, more preferably still at least 95% by weight, and most preferably at least 98% by weight, of the same molecule is present. In some embodiments, the term “purified” means at least 75% by weight, at least 76% by weight, at least 77% by weight, at least 78% by weight, at least 79% by weight, at least 80% by weight, at least 81% by weight, at least 82% by weight, at least 83% by weight, at least 84% by weight, at least 85% by weight, at least 86% by weight, at least 87% by weight, at least 88% by weight, at least 89% by weight, at least 90% by weight, at least 91% by weight, at least 92% by weight, at least 93% by weight, at least 94% by weight, at least 95% by weight, at least 96% by weight, at least 97% by weight, at least 98% by weight, or at least 99% by weight of the same molecule is present.

The terms “engineered,” “genetically engineered,” “genetically modified,” “recombinant,” “modified,” “non-naturally occurring,” and “non-native” indicate intentional human manipulation of a wild-type nucleic acid or protein. The terms encompass methods of genomic modification that include genomic editing, as well as techniques that alter gene expression or inactivation, enzyme engineering, directed evolution, knowledge-based design, random mutagenesis methods, gene shuffling, codon optimization, and the like. Methods for genetic and protein engineering are known in the art.

“Covalent bond,” “covalently attached,” “covalently bound,” “covalently linked,” “covalently connected,” and “molecular bond” are used interchangeably herein and refer to a chemical bond that involves the sharing of electron pairs between atoms. Examples of covalent bonds include, but are not limited to, phosphodiester bonds and phosphorothioate bonds.

“Non-covalent bond,” “non-covalently attached,” “non-covalently bound,” “non-covalently linked,” “non-covalent interaction,” and “non-covalently connected” are used interchangeably herein, and refer to any relatively weak chemical bond that does not involve sharing of a pair of electrons. Multiple non-covalent bonds often stabilize the conformation of macromolecules and mediate specific interactions between molecules. Examples of non-covalent bonds include, but are not limited to hydrogen bonding, ionic interactions (e.g., Na⁺Cl⁻), van der Waals interactions, and hydrophobic bonds.

As used herein, “sequence identity” generally refers to the percent identity of nucleotide bases or amino acids comparing a first polynucleotide or polypeptide to a second polynucleotide or polypeptide using algorithms having various weighting parameters. Sequence identity between two polynucleotides or two polypeptides can be determined using sequence alignment by various methods and computer programs (e.g., BLAST, CS-BLAST, FASTA, HMMER, L-ALIGN, and the like) available through the worldwide web at sites including, but not limited to, GENBANK (www.ncbi.nlm.nih.gov/genbank/) and EMBL-EBI (www.ebi.ac.uk.). Sequence identity between two polynucleotides or two polypeptide sequences is generally calculated using the standard default parameters of the various methods or computer programs. A high degree of sequence identity between two polynucleotides or two polypeptides is typically between about 90% identity and 100% identity over the length of the reference polypeptide, for example, about 90% identity or higher, preferably about 95% identity or higher, more preferably about 98% identity or higher. A moderate degree of sequence identity between two polynucleotides or two polypeptides is typically between about 80% identity to about 85% identity, for example, about 80% identity or higher, preferably about 85% identity over the length of the reference polypeptide. A low degree of sequence identity between two polynucleotides or two polypeptides is typically between about 50% identity and 75% identity, for example, about 50% identity, preferably about 60% identity, more preferably about 75% identity over the length of the reference polypeptide.

For instance, a sequence of the present disclosure may have a particular sequence identity to a reference sequence. This sequence identity may be, for example, 25% or more, 50% or more, 75% or more, 80% or more, 85% or more, 90% or more, 91% or more, 92% or more, 93% or more, 94% or more, 95% or more, 96% or more, 97% or more, 98% or more, or 99% or more.

As used herein, “binding” refers to a non-covalent interaction between macromolecules (e.g., between a protein and a polynucleotide, between a polynucleotide and a polynucleotide, or between a protein and a protein, and the like). Such non-covalent interaction is also referred to as “associating” or “interacting” (e.g., if a first macromolecule interacts with a second macromolecule, the first macromolecule binds to second macromolecule in a non-covalent manner). Some portions of a binding interaction may be sequence-specific (the terms “sequence-specific binding,” “sequence-specifically bind,” “site-specific binding,” and “site specifically binds” are used interchangeably herein). Binding interactions can be characterized by a dissociation constant (Kd). “Binding affinity” refers to the strength of the binding interaction. An increased binding affinity is correlated with a lower Kd.

A “coding sequence” or a sequence that “encodes” a selected polypeptide, is a nucleic acid molecule that is transcribed (in the case of DNA) and translated (in the case of mRNA) into a polypeptide in vitro or in vivo when placed under the control of appropriate regulatory sequences. The boundaries of the coding sequence are determined by a start codon at the 5′ terminus and a translation stop codon at the 3′ terminus. A transcription termination sequence may be located 3′ to the coding sequence.

As used herein, the term “modulate” refers to a change in the quantity, degree or amount of a function. Thus, “modulation” of gene expression includes both gene activation and gene repression. Modulation can be assayed by determining any characteristic directly or indirectly affected by the expression of the target gene. Such characteristics include, for example, changes in RNA or protein levels, protein activity, product levels, expression of the gene, or activity level of reporter genes.

As used herein, the term “between” is inclusive of end values in a given range (e.g., between 10 and 50 amino acids in length includes 10 amino acids and 50 amino acids).

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms also refer to an amino acid polymer that has been modified through, for example, acetylation, disulfide bond formation, glycosylation, lipidation, phosphorylation, pegylation, biotinylation, cross-linking, and/or conjugation (e.g., with a labeling component or ligand). Polypeptide sequences are displayed herein in the conventional N-terminal to C-terminal orientation, unless otherwise indicated. Polypeptides and polynucleotides can be made using routine techniques in the field of molecular biology.

The terms “fusion protein” and “chimeric protein” as used herein refer to a single protein created by joining two or more proteins, protein domains, or protein fragments that do not naturally occur together in a single protein. This joining may be direct, e.g., no intervening sequence (such as a linker sequence); or indirect, e.g., containing one or more linker sequences.

As used herein, the term “linked” refers to the joining of two or more components, cither directly and/or indirectly. For example, a first moiety may be covalently or noncovalently (e.g., electrostatically) linked to a second moiety. This includes, but is not limited to, covalently bonding one molecule to another molecule, noncovalently bonding one molecule to another (e.g., electrostatically bonding), non-covalently bonding one molecule to another molecule by hydrogen bonding, non-covalently bonding one molecule to another molecule by van der Waals forces, and any and all combinations of such couplings. Indirect attachment is possible, such as by using a “linker” (a molecule or group of atoms positioned between two moieties). In the present disclosure, a linker may be a peptide linker sequence that, for example, reduces steric hindrance. For example, a fusion peptide may comprise two peptide regions that are directly linked to each other, or indirectly linked to each other by the presence of an intervening linker.

A “moiety” as used herein refers to a portion of a molecule. A moiety can be a functional group or describe a portion of a molecule with multiple functional groups (e.g., that share common structural aspects). The terms “moiety” and “functional group” are typically used interchangeably; however, a “functional group” can more specifically refer to a portion of a molecule that comprises some common chemical behavior. “Moiety” is often used as a structural description.

The terms “modified protein,” “mutated protein,” “protein variant,” and “engineering protein” as used herein typically refers to a protein that has been modified such that it comprises a non-native sequence (i.e., the modified protein has a unique sequence compared to an unmodified protein); or a native sequence but with one or more non-native modifications, conjugations, etc.

As used herein, the term “antigen” means any antigen that can generate one or more immune responses. The antigen may be one that generates a humoral and/or a cytotoxic T-lymphocyte (CTL) immune response, for example.

As used herein, the term “antigen-presenting cell” or “APC” refers to a cell that can present antigen bound to MHC class I or class II molecules to T-cells. APCs include, but are not limited to, monocytes, macrophages, dendritic cells, B-cells, T-cells and Langerhans cells. A T-cell that can present antigen to other T cells (including CD4+ and/or CD8+ T cells) is an antigen presenting T-cell (T-APC). Fragments or portions of antigens herein may be presented on APCs, for example.

As used herein, the term “adjuvant” refers to any material added to a vaccine to enhance the immunogenicity of an antigen.

As used herein, the term “immune response” refers to a response of a cell of the immune system, such as a B-cell, T-cell, or monocyte, to a stimulus. The response may be specific for a particular antigen (an “antigen-specific response”). An immune response may involve or primarily be, for example, a T-cell response (such as a CD4+ response or a CD8+ response); or a B-cell response (resulting in the production of specific antibodies). A generated immune response may also be characterized, for example, as being a Th1 or Th2 immune response; a predominantly Th1 or Th2 immune response; or a mixed Th1 or Th2 immune response.

As used herein, the term “immunogenic composition” refers to a composition that contains at least one antigen, and which elicits an immune response in the host (when administered to the host). An “immune response” may include, but is not limited to, one or more of the following: antibody production and/or class switching, a B-cell response, a helper T-cell response, a suppressor T-cell response, cytokine and/or inflammatory mediator production, a cytotoxic T-cell response, a gamma-delta T-cell response, induction and/or maturation of dendritic cells, and interferon (Type I or II) synthesis, for example. Ideally, the generated immune response in the host will provide a protective immune response so that susceptibility to infection will be reduced, and/or the clinical severity of the infection will be reduced. Such a protective immune response may be manifested by, for example, a reduction or lack of clinical signs or symptoms typically displayed by an infected host; a reduced viral load; a quicker recovery time; a lowered duration of symptoms; and/or a lowered duration of viral shedding.

As used herein, the term “immunogenic fragment” refers to a fragment or portion of a polypeptide, or a nucleotide sequence encoding the same, which an immune response may be generated against. The fragment encompasses truncated fragments. In some embodiments, the truncated fragments are obtained from full-length proteins by deleting a transmembrane sequence(s).

The terms “subject,” “individual.” or “patient” are used interchangeably herein and refer to any member of the phylum Chordata, including, without limitation, humans and other primates, including non-human primates, such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like. The term does not denote a particular age or gender. Thus, the term includes adult, young, and newborn individuals as well as males and females.

The terms “effective amount” or “therapeutically effective amount” of a composition or agent, refer to a sufficient amount of the composition or agent to provide the desired response. Preferably, the effective amount will prevent, avoid, or eliminate one or more harmful side-effects. The exact amount required will vary from subject to subject, depending on the species, age, and general condition of the subject, the mode of administration, and the like. An appropriate “effective” amount in any individual case may be determined by one of ordinary skill in the art using routine experimentation.

The term “poxvirus” refers to viruses belonging to the family Poxviridae. Members of the Chordopoxviriniae subfamily infect vertebrates, and include, without limitation, viruses within the genera Avipoxvirus (e.g., canarypoxvirus virus and fowlpox virus), Capripoxvirus (e.g., Sheeppox virus, Goatpox virus and Lumpy skin disease virus), Centapoxvirus, Cervidpoxvirus, Crocodylidpoxvirus, Leporipoxvirus (e.g., Myxoma virus, Shope fibroma virus, Squirrel fibroma virus and Hare fibroma virus), Macropopoxvirus, Molluscipoxvirus (e.g., Molluscum contagiosum virus), Mustelpoxvirus, Orthopoxvirus (e.g., Camelpox virus, Cowpox virus, Ectromelia virus, Horsepox virus, Monkeypox virus, Raccoonpox virus, Skunkpox virus, Taterapox virus, Uasin Gishu virus, Vaccinia virus, Variola virus and Volepox virus), Oryzopoxvirus, Parapoxvirus (e.g., Bovine papular stomatitis virus and Orf virus), Pteropopoxvirus, Salmonpoxvirus, Suipoxvirus (e.g., Swinepox virus), Vespertilionpoxvirus and Yatapoxvirus (e.g., Tanapox virus and Yaba monkey tumor virus).

As used herein, the term “vaccine” refers to a composition or product that elicits a prophylactic or therapeutic immune response in a subject. In some cases, the immune response is a protective immune response.

As used herein, the term “virus-like particle” or “VLP” refers to a non-replicating, viral protein shell. VLPs are generally composed of one or more viral proteins, such as, but not limited to, viral proteins such as capsid, coat, shell, surface and/or envelope proteins (or polypeptides or fragments thereof) derived from such proteins. VLPs may form spontaneously upon recombinant expression of the protein(s) in an appropriate expression system, or when induced by a suitable inducer.

### Poxvirus Protein Antigens

The present disclosure provides nanoparticle-based vaccines and vaccine compositions which comprise nanoparticles linked to one or more antigens. In some embodiments thereof, the one or more antigens are proteins (or fragments, portions, variants or derivatives thereof) from a poxvirus. In some embodiments thereof, the poxvirus is an orthopoxvirus. In further embodiments thereof, the orthopoxvirus may be selected from the group consisting of a vaccinia virus, a monkeypox virus, and a variola virus.

In some embodiments, the one or more antigens are proteins (or fragments, portions, variants or derivatives thereof) from the same or from different poxviruses, such as, for example, a vaccinia virus, a monkeypox virus, and a variola virus.

In some embodiments of nanoparticle-based vaccines and vaccine compositions of the present disclosure, nanoparticles may be linked to at least 1, at least 2, at least 3, at least 4, at least 5, at least 6, at least 7, at least 8, at least 9, or at least 10, different poxvirus proteins (or fragments, portions, variants or derivatives thereof).

In some embodiments, the at least 2, at least 3, at least 4, at least 5, at least 6, at least 7, at least 8, at least 9, or the at least 10 different proteins (or fragments, portions, variants or derivatives thereof) are from the same poxvirus. In some embodiments, the at least 2, at least 3, at least 4, at least 5, at least 6, at least 7, at least 8, at least 9, or the at least 10 different proteins (or fragments, portions, variants or derivatives thereof) may originate from two or more, three or more, four or more, or five or more, different poxviruses.

In some embodiments, a nanoparticle is attached to at least one membrane-associated protein (or fragment, portion, variant or derivative thereof) from the membrane of a poxvirus mature virion (MV). MV may undergo additional membrane wrapping by a trans-Golgi or endosomal cisterna that has been modified by the insertion of viral proteins, which upon exocytosis at the cellular plasma membrane, releases extracellular virions (EVs). The EV can be thought of as consisting of an MV surrounded by one additional membrane wrapper, though there are differences between an MV released by lysis of cells and disruption of the EV wrapper. In some embodiments, a nanoparticle is attached to at least one membrane-associated protein from the EV membrane. Bernard Moss, Poxvirus entry and membrane fusion, Virology, 344 (2006), pp. 48-54.

In some embodiments, a nanoparticle is attached to at least one poxvirus MV protein (or fragment, portion, variant or derivative thereof) selected from vaccinia virus A26, A27, D8, H3, A16, A21, A28, F9, G3, G9, H2, J5, L1, L5, O3, A2.5, E10, G4, A9, A13, A14, A17, I2, A14.5, F14.5, and I5 or orthopoxvirus homologs. In some embodiments, a nanoparticle is attached to at least one poxvirus EV protein (or fragment, portion, variant or derivative thereof) selected from vaccinia virus A33, A34, A56, B5, F13, and K2 or orthopoxvirus homologs. In some embodiments, two or more poxvirus MV proteins as described herein may form a fusion protein or a multimer.

In some embodiments, a nanoparticle is attached to at least one poxvirus protein (or fragment, portion, variant or derivative thereof) selected from L1, M1 (homolog of L1), A27, A28, H3, D8, A13, A17, A33, A35 (homolog of A33), B5, and B6 (homolog of B5). In some embodiments thereof, the nanoparticle is attached to at least 1, at least 2, at least 3, at least 4, at least 5, at least 6, at least 7, at least 8, or 9, poxvirus proteins (or fragment, portion, variant or derivative thereof) selected from vaccinia virus L1, A27, A28, H3, D8, A13, A17, A33, H2, B5, A16, G9 or monkeypox virus homologs. In some embodiments thereof, the nanoparticle is attached to at least, or only, poxvirus proteins (or fragments, portions, variants or derivatives thereof) vaccinia virus L1, A33, A35, B5, A28, and H2 or monkeypox M1, A35, B6, A30 or H2. In some embodiments thereof, the nanoparticle is attached to at least, or only, poxvirus proteins (or fragments, portions, variants or derivatives thereof) vaccinia virus L1, A33, B5, or monkeypox M1, A35 or B6. In some embodiments, two or more poxvirus MV proteins as described herein may form a fusion protein or a multimer.

The above protein designations are based on the Vaccinia virus Copenhagen or conventional monkeypox virus nomenclature, and it is readily apparent and known in the art what the corresponding homologous gene sequences are in other poxviruses, including, without limitation, monkeypox virus and variola virus; as such, it will be appreciated that these listed MV- and EV-membrane proteins, in different embodiments, may originate from the same or from different poxviruses. Accordingly, unless otherwise noted, the Vaccinia virus Copenhagen nomenclature is used to also denote corresponding genes in other poxviruses.

In some embodiments, an antigen may be a variant of a wild-type poxvirus protein that has one or more (e.g., at least 1, 2, 3, 4, 5, 6, 7, 8, 9, 10, 20, 30, 40, 50, etc.) amino acid additions, deletions, or substitutions, as compared to the corresponding region in the wild-type poxvirus protein. In some embodiments thereof, a variant protein has 80% or more, 81% or more, 82% or more, 83% or more, 84% or more, 85% or more, 86% or more, 87% or more, 88% or more, 88% or more, 90% or more, 91% or more, 92% or more, 93% or more, 94% or more, 95% or more, 96% or more, 97% or more, 98% or more, or 99% or more, identity to the corresponding region in the wild-type poxvirus protein.

In some embodiments, some or all of the different poxvirus proteins (or fragments, portions, or derivatives thereof) may be fused, directly or indirectly (e.g., separated by an intervening linker sequence(s)), to each other (e.g., by covalent bonding) to produce a fusion or chimeric protein(s) that is then bound to a nanoparticle. In some embodiments thereof, poxvirus proteins (or fragments, portions, or derivatives thereof) A28 and H2 are linked to each other, and this fusion or chimeric protein is then bound to a nanoparticle.

In some embodiments, the fragment or portion of a poxvirus protein comprises a C-terminal fragment or portion. In some embodiments, the fragment or portion of a poxvirus protein comprises an N-terminal fragment or portion. In some embodiments, the fragment or portion of a poxvirus protein comprises an internal fragment or portion.

In some embodiments, the fragment or portion is at least 10 amino acids, at least 20 amino acids, at least 50 amino acids, at least 100 amino acids, at least 150 amino acids, at least 200 amino acids, or at least 250 amino acids, in length.

In some embodiments, some or all of the different poxvirus proteins (or fragments, portions, or derivatives thereof) may contain one or more non-naturally occurring amino acids, and/or one or more modified amino acids. In some embodiments thereof, the modification may include, for example, acetylation, disulfide bond formation, glycosylation, lipidation, phosphorylation, pegylation, biotinylation, cross-linking, and/or conjugation (e.g., with a labeling component or ligand).

In some embodiments, a fragment or portion of a poxvirus protein may be used which lacks a part or the entirety of the transmembrane domain(s) of the native, full-length protein.

In some embodiments, one or more poxvirus proteins (or fragments, portions, or derivatives thereof) may be linked, directly or indirectly (e.g., by covalent bonding), to one or more of a peptide tag, small molecule, moiety, or binding domain, that facilitates binding to a nanoparticle.

Exemplary peptide tags for facilitating binding to a nanoparticle include, but are not limited to: AviTag (GLNDIFEAQKIEWHE (SEQ ID NO: 1), which allows biotinylation by BirA, and thereafter, binding by streptavidin); Calmodulin-tag (KRRWKKNFIAVSAANRFKKISSSGAL (SEQ ID NO: 2), which binds calmodulin); S-tag (KETAAAKFERQHMDS (SEQ ID NO: 3), which binds to S-protein); SBP-tag (MDEKTTGWRGGHVVEGLAGELEQLRARLEHHPQGQREP (SEQ ID NO: 4), which binds to streptavidin); Strep-tag II (WSHPQFEK (SEQ ID NO: 5), which binds to streptavidin or streptactin); Isopeptag (TDKDMTITFTNKKDAE (SEQ ID NO. 6), which binds covalently to pilin-C protein); Spytag (AHIVMVDAYKPTK (SEQ ID NO: 7), which binds to SpyCatcher protein) and polyhistidine tag, which binds to Ni-NTA.

In some embodiments, one or more poxvirus proteins (or fragments, portions, or derivatives thereof) may be linked, directly or indirectly (e.g., by covalent bonding), to one or more of a peptide tag, small molecule, moiety, or binding domain, that facilitates purification of the expressed poxvirus protein prior to attachment to the nanoparticle. Exemplary peptide tags for facilitating purification of the poxvirus protein prior to attachment to the nanoparticle include, but are not limited to: AviTag (GLNDIFEAQKIEWHE (SEQ ID NO: 1); which allows biotinylation by BirA, and thereafter, binding by streptavidin); Calmodulin-tag (KRRWKKNFIAVSAANRFKKISSSGAL (SEQ ID NO: 2); which binds calmodulin); S-tag (KETAAAKFERQHMDS (SEQ ID NO: 3); which binds to S-protein); SBP-tag (MDEKTTGWRGGHVVEGLAGELEQLRARLEHHPQGQREP (SEQ ID NO: 4); which binds to streptavidin); Strep-tag II (WSHPQFEK (SEQ ID NO: 5); which binds to streptavidin or streptactin); Isopeptag (TDKDMTITFTNKKDAE (SEQ ID NO: 6); which binds covalently to pilin-C protein); Spytag (AHIVMVDAYKPTK (SEQ ID NO: 7); which binds to SpyCatcher protein); poly-histidine (poly(His)), chitin binding protein, maltose binding protein, glutathione-S-transferase (GST), FLAG-tag, E-tag, HA-tag, Myc-tag, Softtag 1, Softtag 3, TC tag, V5 tag, VSV tag, Xpress tag, Biotin Carboxyl Carrier Protein (BCCP) tag, Halo-tag, thioredoxin-tag, or Fc-tag.

SpyCatcher-SpyTag refers to a protein ligation system that is based on the internal isopeptide bond of the CnaB2 domain of FbaB, a fibronectin-binding MSCRAMM and virulence factor of Streptococcus pyogenes. It utilizes a modified domain from a Streptococcus pyogenes surface protein (SpyCatcher), which recognizes a cognate 13-amino-acid peptide (SpyTag). Upon recognition, the two form a covalent isopeptide bond between the side chains of a lysine in SpyCatcher, and an aspartate in SpyTag.

### Nanoparticles

The present disclosure provides nanoparticle-based vaccines and vaccine compositions which comprise nanoparticles linked, directly or indirectly, to one or more antigens. In some embodiments thereof, an antigen (e.g., a poxvirus protein, or fragment, portion, variant or derivative thereof) is linked to a nanoparticle via a peptide tag, small molecule, moiety, or binding domain, that facilitates binding to a target motif on a nanoparticle.

For instance, a poxvirus protein (or fragment, portion, variant or derivative thereof) may be linked, directly or indirectly, to a peptide tag, small molecule, moiety, or binding domain that binds a target motif present on the nanoparticle. In some embodiments, the target motif to which the peptide tag, small molecule, moiety, or binding domain, binds is linked, directly or indirectly, to a part of the nanoparticle. In some embodiments, the target motif is linked, directly or indirectly, to a polypeptide that constitutes a part or monomer of the nanoparticle.

For instance, in some embodiments, the nanoparticle contains, as a part thereof (e.g., directly or indirectly linked to a scaffold component of the nanoparticle) streptavidin (as a targeting motif), which then facilitates binding to a poxvirus protein (or fragment, portion, variant or derivative thereof) that is linked, directly or indirectly, to an AviTag (GLNDIFEAQKIEWHE (SEQ ID NO: 1)). In some embodiments, the nanoparticle contains, as a part thereof (e.g., directly or indirectly linked to a scaffold component of the nanoparticle) calmodulin (as a targeting motif), which then facilitates binding to a poxvirus protein (or fragment, portion, variant or derivative thereof) that is linked, directly or indirectly, to a Calmodulin-tag (KRRWKKNFIAVSAANRFKKISSSGAL (SEQ ID NO: 2)). In some embodiments, the nanoparticle contains, as a part thereof (e.g., directly or indirectly linked to a scaffold component of the nanoparticle) S-protein (as a targeting motif), which then facilitates binding to a poxvirus protein (or fragment, portion, variant or derivative thereof) that is linked, directly or indirectly, to an S-tag (KETAAAKFERQHMDS (SEQ ID NO: 3)). In some embodiments, the nanoparticle contains, as a part thereof (e.g., directly or indirectly linked to a scaffold component of the nanoparticle) streptavidin (as a targeting motif), which then facilitates binding to a poxvirus protein (or fragment, portion, variant or derivative thereof) that is linked, directly or indirectly, to an SBP-tag (MDEKTTGWRGGHVVEGLAGELEQLRARLEHHPQGQREP (SEQ ID NO: 4)). In some embodiments, the nanoparticle contains, as a part thereof (e.g., directly or indirectly linked to a scaffold component of the nanoparticle) streptavidin and/or streptactin (as a targeting motif), which then facilitates binding to a poxvirus protein (or fragment, portion, variant or derivative thereof) that is linked, directly or indirectly, to a Strep-tag II (WSHPQFEK (SEQ ID NO: 5)). In some embodiments, the nanoparticle contains, as a part thereof (e.g., directly or indirectly linked to a scaffold component of the nanoparticle) pilin-C protein (as a targeting motif), which then facilitates binding to a poxvirus protein (or fragment, portion, variant or derivative thereof) that is linked, directly or indirectly, to an Isopeptag (TDKDMTITFTNKKDAE (SEQ ID NO: 1) (SEQ ID NO: 6)). In some embodiments, the nanoparticle contains, as a part thereof (e.g., directly or indirectly linked to a scaffold component of the nanoparticle) a SpyCatcher protein (as a targeting motif), which then facilitates binding to a poxvirus protein (or fragment, portion, variant or derivative thereof) that is linked, directly or indirectly, to a Spytag (AHIVMVDAYKPTK (SEQ ID NO: 7)).

In some embodiments, the targeting motif to which the peptide tag, small molecule, moiety, or binding domain (present on the antigen) binds is linked, directly or indirectly, to a polypeptide that constitutes a monomer of a self-assembling protein nanoparticle. In some embodiments, the nanoparticle is formed or derived from a virus-like particle (VLP).

Alternatively, in some embodiments, the above-described targeting motifs are linked, directly or indirectly, to a poxvirus antigen; and a cognate peptide tag, small molecule, moiety, or binding domain (e.g., AviTag, Calmodulin-tag, S-tag, SBP-tag, Strep-tag II, Isopeptag, Spytag, etc.) is present on the nanoparticle.

The present disclosure encompasses any nanoparticle scaffold suitable for antigen attachment, presentation, and/or delivery. This includes, without limitation, virus-like particles (VLP) such as bacteriophages Qbeta and AP205, Hepatitis B virus, adeno-associated virus, HIV, Hepatitis C virus, Chikungunya virus, Equine encephalitis virus, Nipah virus, Human papillomavirus, Flock House Virus, ORSAY virus, and infectious bursal disease virus, etc.

In some embodiments, the nanoparticles are self-assembling nanoparticles derived from ferritin (FR), E2p, I3-01, lumazine synthase, encapsulin, influenza matrix 1 protein, dihydrolipoyl acetyltransferase, or coronavirus nonstructural protein 1.

In embodiments of the present disclosure, any of these known nanoparticles can be utilized, as well as modified variants thereof (e.g., having at least 90%, 91%, 92%, 93%, 94%, 95%, 96%, 97%, 98%, or 99% identity to a native wild-type sequence). In some embodiments, a component or monomer of the nanoparticle (such as a polypeptide monomer) is further linked, directly or indirectly (e.g., by covalent bonding), to one or more of a peptide tag, small molecule, moiety, or binding domain, that facilitates purification of the nanoparticle component or monomer prior to attachment to the antigen(s). Exemplary peptide tags for facilitating purification of the nanoparticle component or monomer prior to attachment to the antigen(s) include, but are not limited to: AviTag (GLNDIFEAQKIEWHE (SEQ ID NO: 1); which allows biotinylation by BirA, and thereafter, binding by streptavidin); Calmodulin-tag (KRRWKKNFIAVSAANRFKKISSSGAL (SEQ ID NO: 2); which binds calmodulin); S-tag (KETAAAKFERQHMDS (SEQ ID NO: 3); which binds to S-protein); SBP-tag (MDEKTTGWRGGHVVEGLAGELEQLRARLEHHPQGQREP (SEQ ID NO: 4); which binds to streptavidin); Strep-tag II (WSHPQFEK (SEQ ID NO: 5); which binds to streptavidin or streptactin); Isopeptag (TDKDMTITFTNKKDAE (SEQ ID NO: 6); which binds covalently to pilin-C protein); Spytag (AHIVMVDAYKPTK (SEQ ID NO: 7); which binds to SpyCatcher protein); poly-histidine (poly(His)), chitin binding protein, maltose binding protein, glutathione-S-transferase (GST), FLAG-tag, E-tag, HA-tag, Myc-tag, Softtag 1, Softtag 3, TC tag, V5 tag, VSV tag, Xpress tag, Biotin Carboxyl Carrier Protein (BCCP) tag, Halo-tag, thioredoxin-tag, or Fc-tag.

Nanoparticles can be in an average size range of about 20 nm to about 1000 nm in diameter, such as about 50 nm to about 1000 nm in diameter. The nanoparticles can have an average diameter about 20 nm, about 50 nm, about 75 nm, about 100 nm, about 150 nm, about 200 nm, about 250 nm, about 300 nm, about 400 nm, about 500 nm, about 750 nm, about 1000 nm, or can have an average diameter range of from about 20 nm to about 500 nm, from about 50 nm to about 500 nm, from about 100 nm to about 500 nm, from about 100 nm to about 750 nm, from about 100 nm to about 1000 nm, from about 250 nm to about 750 nm, from about 500 nm to about 1000 nm, from about 250 nm to about 500 nm.

### Nanoparticle-Based Vaccine Compositions

As described herein, nanoparticle-based vaccines and vaccine compositions of the present disclosure can be generated by producing one or more antigens that bind to a nanoparticle, and binding the one or more antigens to the nanoparticle.

In some embodiments, nanoparticles and antigens are synthesized and/or produced in the same reaction. In some embodiments, nanoparticles and antigens are synthesized and/or produced separately, followed by binding the one or more antigens to the nanoparticle. It will be appreciated that nanoparticles may be labeled with a single antigen (homotypic nanoparticles) or multiple different antigens (mosaic nanoparticles), e.g., by generating nanoparticles containing a plurality of different targeting motifs; and/or by using mixtures of different antigens in appropriate proportions, for example.

In some embodiments, a poxvirus protein (or fragment, portion, variant or derivative thereof) to be used as an antigen is expressed by initially removing all or part of the transmembrane domain(s), and attaching a signal sequence (to facilitate solubilization and secretion of the protein).

In some embodiments, nanoparticles, antigens, or antigen-labeled nanoparticles, may be further processed and/or purified by one or more techniques such as centrifugation, ultracentrifugation, filtration, ultrafiltration, gravity, sonication, density-gradient ultracentrifugation, tangential flow filtration, size-exclusion chromatography, ion-exchange chromatography, affinity capture, polymer-based precipitation, or organic solvent precipitation, for example.

Antigens and/or nanoparticle polypeptide components or monomers may be produced by first generating expression constructs (i.e., expression vectors) that encode the antigens and/or nanoparticle polypeptide components or monomers. Such expression vectors can readily be generated using standard molecular biology techniques. For example, general protocols for cloning, transfecting, transient gene expression and obtaining stable transfected cell lines are described in the art, e.g., Sambrook et al., Molecular Cloning: A Laboratory Manual, Cold Spring Harbor Press, N.Y., (3.sup.rd ed., 2000); and Brent et al., Current Protocols in Molecular Biology, John Wiley & Sons, Inc. (ringbou ed., 2003).

In some embodiments, nucleotide sequences encoding an antigen and/or a nanoparticle component or monomer may be optimized (“codon-optimized”) for expression with a particular cell type, such as mammalian cells. In some embodiments, nucleotide sequences encoding an antigen and/or a nanoparticle component or monomer may be modified to remove or introduce a particular post-translational modification site (such as, for example, a site for phosphorylation, acetylation, glycosylation, methylation, or ubiquitination). For instance, N-glycosylation sites may be removed from a protein of interest, such as an L1 protein when used as an antigen in the nanoparticle-based vaccines herein

### Vaccine and Vaccine Compositions

The vaccines and compositions of the present disclosure may further comprise, for example, appropriate adjuvant(s).

Examples of adjuvants include, e.g., aluminum hydroxide, lecithin, Freund's adjuvant, MPL, aluminum phosphate, amorphous aluminum sulphate phosphate, calcium phosphate, inulin, liposomes, chitosan, dextran, dextrins, starch, mannans and glucomannans, galactomannans, beta-glucans, heparin, cellulose, hemicellulose, pectins and pectinates, lectins, polylactide, squalene, saponins (e.g., QS-21, QuilA, tomatine, ISCOMs, ISCOMATRIX etc.), lipopeptides, glycopeptides, resiquimoid, lipopolysaccharides, lipid A, muramyl dipeptides. Immunostimulatory oligonucleotides (such as those including a CpG motif) can also be used as adjuvants. Adjuvants also include biological molecules, such as costimulatory molecules. Exemplary such adjuvants include IL-2, IL-12, RANTES, GM-CSF, TNF-μ, IFN-γ, G-CSF, LFA-3, CD72, B7-1, B7-2, OX-40L, 4-1BBL and Toll-like receptor (TLR) agonists, such as TLR-7/8 agonists.

Vaccine compositions may further comprise a stabilizing agent(s), such as one or more of: lactose, sucrose, trehalose, maltose, mannose, iso-maltose, raffinose, stachyose, lactobiose, sorbitol, mannitol, lactobionic acid, dextran, L-glycine, L-histidine, L-glutamic acid, L-aspartic acid, human serum albumin and combinations thereof.

Additionally, or alternatively, vaccine compositions may be prepared in lyophilized form, such as in the presence of a sugar polyol (such as sorbitol and/or mannitol) and a sugar (e.g., sucrose, trehalose, maltose, mannose, lactose, raffinose, isomaltose, stachyose etc.). Lyophilized formulations can be re-suspended in water or an appropriate aqueous buffer.

Vaccines and vaccine compositions of the present disclosure may be used to induce an immune response against a poxvirus, such as monkeypox virus, in a subject, by administering to the subject a vaccine or vaccine composition as described herein.

The administration of the vaccines and compositions of the present disclosure to subjects may be carried out in any convenient manner, including by aerosol inhalation, injection, ingestion, transfusion, implantation or transplantation. The vaccines and compositions described herein may be administered to a patient subcutaneously, intradermally, intratumorally, intranodally, intramedullary, intramuscularly, by intravenous or intralymphatic injection, or intraperitoneally. The vaccines and compositions can be administered in one or more doses. In some embodiments, an effective amount is administered as a single dose. In other embodiments, an effective amount is administered as more than one dose, over a period time. The period of time between doses can be, for example, at least 1, at least 2, at least 3, at least 4, at least 5, at least 6, at least 7, at least 8, at least 9, at least 10, at least 11, or at least 12, days. The period of time between doses can be, for example, at least 1, at least 2, at least 3, at least 4, at least 5, at least 6, at least 7, at least 8, at least 9, at least 10, at least 11, or at least 12, weeks. The period of time between doses can be, for example, at least 1, at least 2, at least 3, at least 4, at least 5, at least 6, at least 7, at least 8, at least 9, at least 10, at least 11, or at least 12, months. The period of time between doses can be, for example, at least 1, at least 2, at least 3, at least 4, at least 5, at least 6, at least 7, at least 8, at least 9, at least 10, at least 11, or at least 12, years.

### EXAMPLES

### Example 1: Poxvirus Protein Modifications

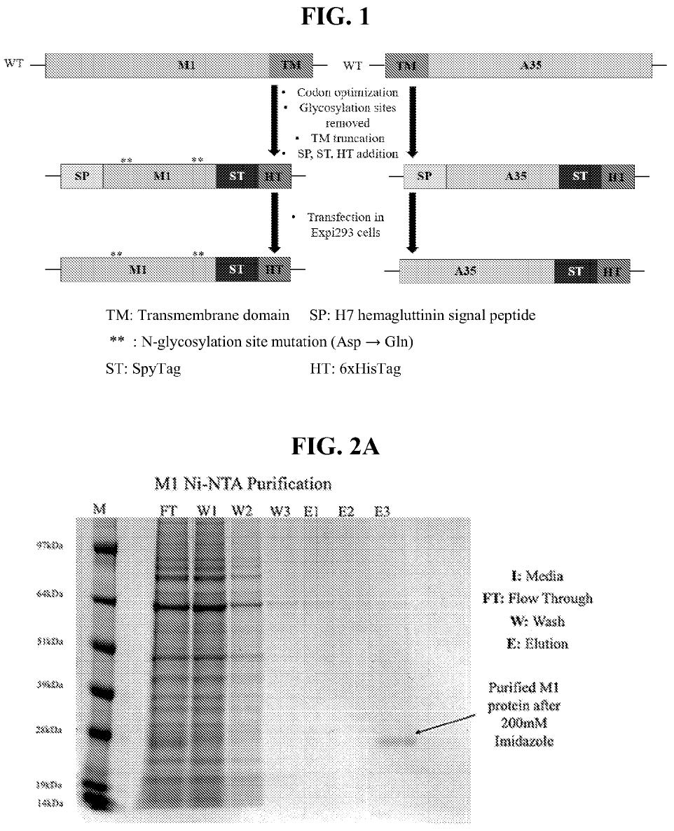

As shown in FIG. 1, genes encoding mpox proteins M1 and A35 proteins were modified through codon optimization, removal of glycosylation sites, truncation of transmembrane domain, and attaching signal peptide (H7 hemagglutinin signal peptide) and tags (SpyTag (ST) and 6×His tag). M1 and A35 proteins used are the Congo Basin monkeypox homologs from Zaire 79 strain.

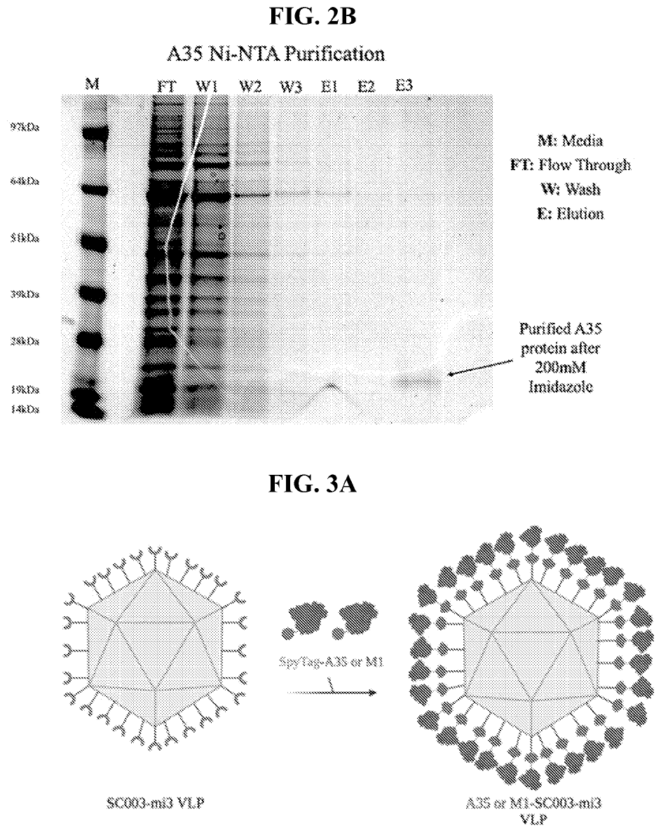

Thus produced genes encoding modified mpox proteins M1 SpyTag and A35 Spy Tag were transfected into Expi293 cells and cultured to express the modified mpox proteins M1 SpyTag and A35 SpyTag. Secreted M1 SpyTag and A35 SpyTag were isolated by one-step purification in which the proteins are bound to Ni-NTA beads and eluted with imidazole. The results are shown in FIG. 2A and FIG. 2B.

### Example 2: Preparation of Nanoparticle-Orthopoxvirus Protein Complex

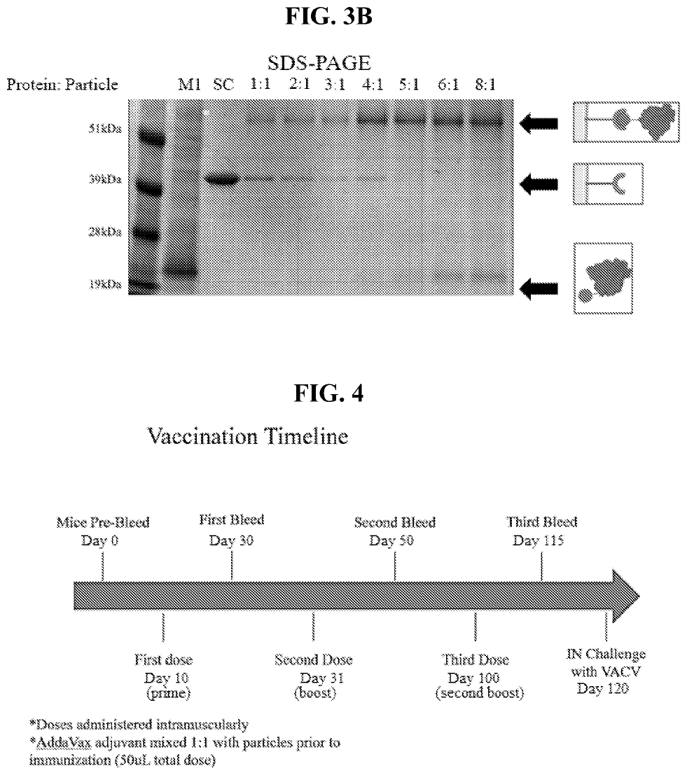

SpyTagged proteins form irreversible isopeptide bond with SpyCatcher particle, as schematically illustrated in FIG. 3A. SpyCatcher003-mi3 virus-like particle (SC003-mi3 VLP) having average 60 binding sites (available from ADDGENE plasmid #159995; n2t.net/addgene: 159995, manufactured by NOVAGEN) were mixed with A35 or M1 SpyTag, obtained in Example 1 at various ratios (M1 or A35 SpyTag protein alone, SpyCatcher003-mi3 alone, or protein: particle ratios of 1:1, 2:1, 3:1, 4:1, 5:1, 5:1, 6:1, or 8:1) to produce nanoparticles to which M1 or A35 proteins are attached irreversibly. SDS-PAGE results of thus formed complexes are shown in FIG. 3B.

### Example 3: Vaccination with Nanoparticle Complexes

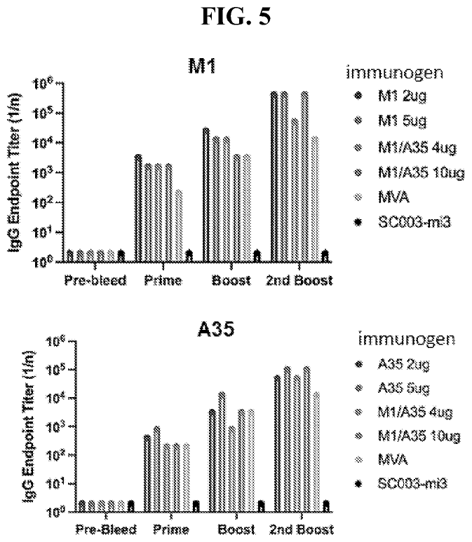

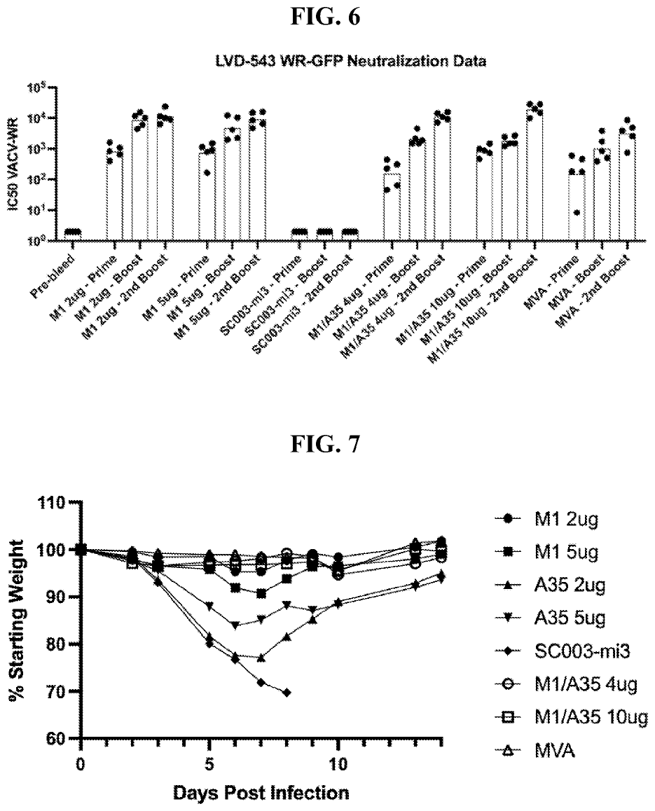

By following the vaccination timeline schedule of FIG. 4, 2 μg of M1-nanoparticle complex or A35-nanoparticle complex, 5 μg of M1-nanoparticle complex or A35-nanoparticle complex, a mixture of 2 μg M1-nanoparticle complex and 2 μg A35-nanoparticle complex, a mixture of 5 μg M1-nanoparticle complex and 5 μg A35-nanoparticle complex, 10⁷ plaque forming units of modified vaccinia Ankara (MVA) (component of Jynneos vaccine) mpox vaccine or 5 μg SpyCatcher003-mi3 (SC003-mi3) were injected into mice, and blood samples were taken at pre-bleed, first dose (prime), second dose (boost) and third dose (second boost), respectively, to measure IgG endpoint titer (FIG. 5) and vaccinia virus Western Reserve strain (VACV-WR)-neutralizing activity (IC₅₀) (FIG. 6).

Over the 15 days period after the VACV challenge (infecting the mice with VACV), the body weight changes were measured and the results are shown in FIG. 7.

The results demonstrate that animals immunized with the orthopoxvirus protein-nanoparticle complexes developed antibodies that bound to immobilized vaccinia virus proteins. And, sera from the mice immunized with the one or more orthopoxvirus protein-nanoparticle complexes according to the present disclosure developed higher neutralizing antibodies than the existing MVA (Jynneos) mpox vaccine to vaccinia virus MVs, indicating that the inventive nanoparticle-orthopoxvirus protein complexes exhibit enhanced neutralizing activity against virus infections and protective action of the host by reducing or preventing weight loss upon lethal vaccinia virus challenge. This results indicate the superiority of the nanoparticle-orthopoxvirus protein complexes because individual soluble orthopoxvirus proteins (e.g., L1 and A33 proteins) did not prevent lethality unless combined.

### Example 4: Orthopoxvirus Proteins-Nanoparticle Complexes

By following the procedure of Examples 1-3, each of vaccinia proteins A28, H2, A16 and G9 or monkeypox virus homologs are modified to add SpyTag. The resulting modified proteins, singly and in different combinations (A28 and H2; A16 and G9; A28, H2, A16, and G9, A28, H2, and A16; A28, H2, and G9; H2, A16 and G9; A28, A16, and G9; or their respective monkeypox virus homologs), are attached to SpyCatcher nanoparticles, producing poxvirus VLPs. The resulting poxvirus protein(s)-nanoparticle complexes are injected into mice according to the timeline of FIG. 4 and blood samples are taken for assaying IgG endpoint titer and VACV-WR neutralizing activity. Also, the body weight changes of the mice are measured for 14 days after the challenge (infection) injection.

### Example 5: Fusion Orthopoxvirus Protein-Nanoparticle Complexes

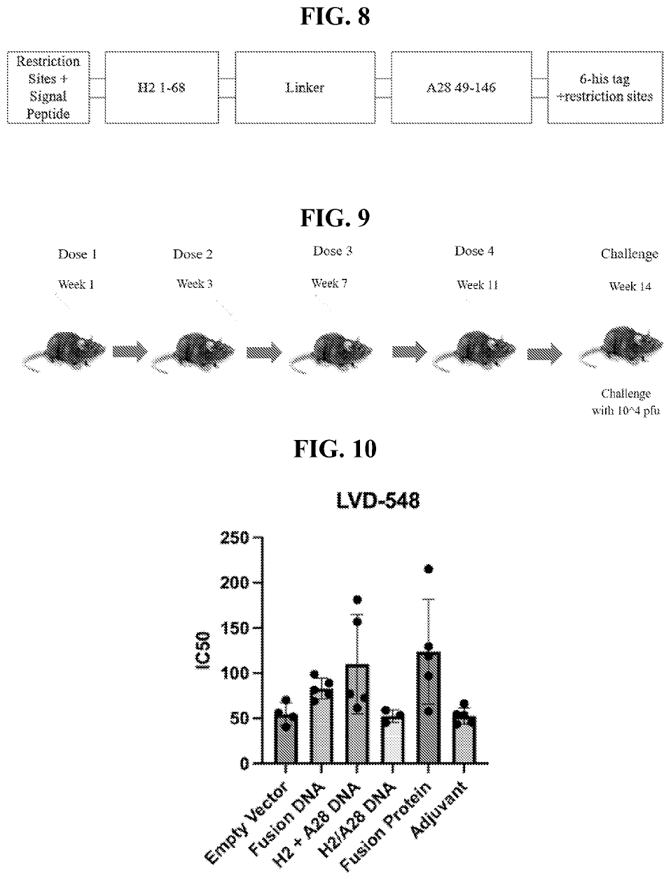

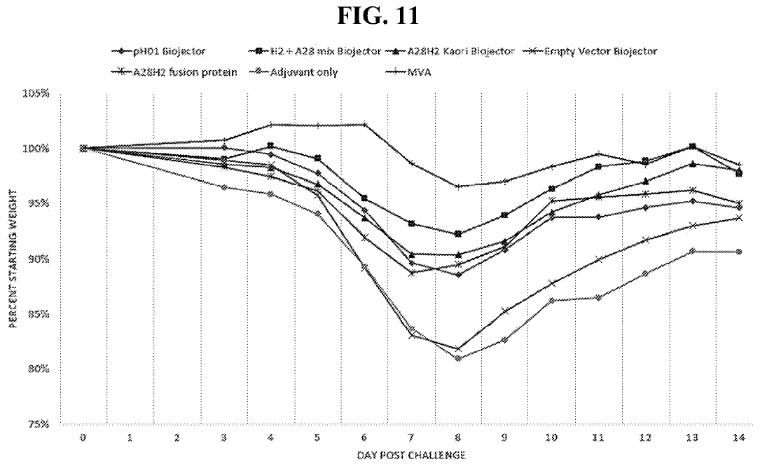

A28 and H2 are two of the nine-reported entry fusion complex (EFC) proteins in VACV. It has been reported that H2 enhanced the effects of A28 when administered together (Shinoda et al. 2010). We produced a fusion protein of truncated A28 and H2, which are linked to each other via a linker (See, FIG. 8). In this non-limiting experiment, transmembrane sequences of A28 and H2 were deleted. To test VACV neutralizing effects and protective activities 10 μg of the resulting fusion protein and adjuvant in comparison with a mixture of 5 μg of A28 and 5 μg of H2 DNAs, 10 μg of A28-H2 fusion protein-encoding DNA, 10 μg empty vector were administered to mice according to the schedule of FIG. 9. The vaccinia virus neutralizing effects (as measured IC₅₀ against VACV-WR) and protective effects against VACV-WR infection (as measured suppressing weight loss) are shown in FIG. 10 and FIG. 11, respectively.

The A28-H2 fusion protein prepared above is modified to add SpyTag by following the procedure of Examples 1-3. The resulting modified fusion protein, singly and in combination with other orthopoxvirus proteins (e.g., A16, G9, A33, L1, or combinations thereof), is attached to VLP nanoparticles with SpyCatcher, producing orthopoxvirus fusion protein-nanoparticle complexes. The resulting orthopoxivirus fusion protein-nanoparticle complex is injected into mice according to the timeline of FIG. 4 and blood samples are taken for assaying IgG endpoint titer and VACV-WR neutralizing activity. Also, the body weight changes of the mice are measured for 14 days after the challenge (infection) injection.

### Example 6: Dimeric Orthopoxvirus Protein-Nanoparticle Complex

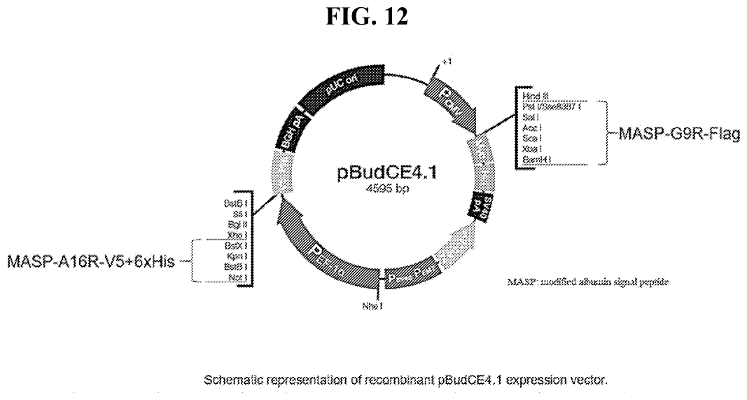

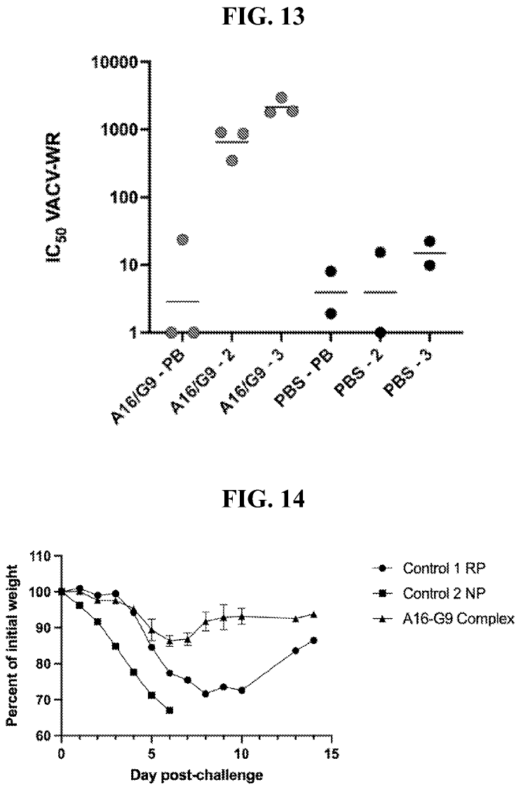

A16 and G9 proteins are essential components of the entry-fusion complex. Full-length A16 and G9 proteins associate to form a dimer. We modeled the protein structure and interaction using the AlphaFold2 prediction program, and generated versions of vaccinia virus A16 and G9 in the transmembrane domains were deleted and signal peptides added and were expressed by Expi293 cells and purified from the medium as a heterodimer. A plasmid pBudCE4.1 containing the truncated A16 and G9R genes, which was used to obtain the A16-G9 heterodimer is illustrated in FIG. 12. Thus-obtained purified A16-G9 heterodimer (10 μg) with adjuvant (Adda Vax) was injected subcutaneously into BALB/c mice three times (day 0, day 21, and day 42) and blood samples were collected before the first injection at day 0, and 2 or 3 weeks after each subsequent injections. When the vaccinia virus neutralizing effects (as measured IC₅₀ against VACV-WR strain) and protective effects against the VACV-WR infection (as measured suppressing weight loss) are shown in FIG. 13 and FIG. 14, respectively. The results of FIG. 13 and FIG. 14 demonstrate that the A16-G9 heterodimer inoculation produces neutralizing antibody against VACV infection and produces noticeable protective effects as shown by the suppressed weight loss after VACV infection. In FIG. 13, prebleed (PB), 2 weeks after the second dose (−2), and 2 weeks after the third dose (−3). And FIG. 14 shows weight loss following infection with VACV WR of the two control mice (RP and NP) that received phosphate buffered saline (PBS) and the mean weight loss of the three mice that received A16-G9 heterodimer.

The A16-G9 heterodimer prepared above is modified to add SpyTag by following the procedure of Examples 1-3. The resulting modified dimeric protein is attached to Spycatcher nanoparticles, producing VLP complexes. The resulting protein-nanoparticle complex is injected into mice according to the timeline of FIG. 4 and blood samples are taken for assaying IgG endpoint titer and VACV-WR neutralizing activity. Also, the body weight changes of the mice are measured for 14 days after the challenge infection.

1. A vaccine composition for eliciting an immune response against a poxvirus, wherein said vaccine composition comprises nanoparticles with at least one poxvirus antigen attached thereto,
wherein said at least one poxvirus antigen is a protein, or fragment, variant, or derivative thereof, from a poxvirus selected from the group consisting of vaccinia virus, monkeypox virus, and variola virus; and
wherein said nanoparticles with at least one poxvirus antigen attached thereto are a virus-like particle (VLP).

2. (canceled)

3. The composition of claim 1, wherein a plurality of different poxvirus antigens is attached to said nanoparticles.

4. The composition of claim 3, wherein the plurality of different poxvirus antigens comprises poxvirus antigens from different poxviruses or from same poxviruses

5. (canceled)

6. The composition of claim 4, wherein the plurality of different poxvirus antigens is from vaccinia virus, monkeypox virus, or combinations thereof.

7. The composition of claim 1, wherein at least three different poxvirus antigens are attached to said nanoparticles.

8. The composition of claim 7, wherein said at least three different poxvirus antigens are proteins, or fragments, variants, or derivatives thereof, of a poxvirus protein selected from L1, A33, B5, A28, H2, A16, and G9 from vaccinia virus, and their respective homologs M1, A35, B6, A30, H2, A17, and G10 from monkeypox virus.

9. (canceled)

10. The composition of claim 7, wherein the at least three different poxvirus antigens comprise L1, A33, and B5 proteins from vaccinia virus and/or their respective homologs M1, A35 and B6 proteins from monkeypox virus, or fragments, variants, or derivatives thereof.

11. The composition of claim 7, wherein the at least three different poxvirus antigens comprise L1, A33, B5, A28, and H2 proteins from vaccinia virus and/or their respective homologs M1, A35, B6, A30, and H2 proteins from monkeypox virus, or fragments, variants, or derivatives thereof.

12. The composition of claim 1, wherein said at least one poxvirus antigen is fused to a peptide tag sequence that facilitates binding to said nanoparticles.

13. The composition of claim 12, wherein said peptide tag sequence is a Spytag sequence.

14. The composition of claim 13, wherein said nanoparticles comprise a SpyCatcher polypeptide, and wherein said tag sequence binds to said SpyCatcher polypeptide.

15. The composition of claim 14, wherein said SpyCatcher polypeptide is fused to a component, monomer, or peptide, of said nanoparticle.

16. (canceled)

17. The composition of claim 1, wherein at least four different poxvirus antigens are attached to said nanoparticles.

18. The composition of claim 1, wherein at least five different poxvirus antigens are attached to said nanoparticles.

19. The composition of claim 1, wherein at least six different poxvirus antigens are attached to said nanoparticles.

20. The composition of claim 17, wherein said different poxvirus antigens are proteins, or fragments, variants, or derivatives thereof, of a poxvirus protein selected from A26, A27, D8, H3, A16, A21, A28, F9, G3, G9, H2, J5, L1, L5, O3, A2.5, E10, G4, A9, A13, A14, A17, I2, A14.5, F14.5, I5, A27, A28, H3, D8, A13, A17, A33, and B5 from vaccinia virus, or their respective homologs from monkeypox virus.

21. A method for eliciting an immune response against an orthopoxvirus in a subject or for immunizing a subject against an orthopoxvirus, comprising administering an effective amount of the composition of claim 1 to the subject.

22. (canceled)

23. The method of claim 21, wherein said administering is by intramuscular or subcutaneous injection.

24. The method of claim 21, wherein the administration of said composition prevents, decreases the severity of, decreases the morbidity of, decreases the mortality of, shortens the duration of, and/or reduces the symptoms of, an orthopoxvirus infection.

25. The composition of claim 1, wherein the at least one poxvirus antigen comprises soluble A16 protein, soluble G9 protein, or a soluble heterodimer of A16/G9 protein.

26. A soluble form of vaccinia virus A16 protein, G9 protein, or A16/G9 heterodimer.

27. A vaccine composition comprising the soluble form of vaccinia virus A16 protein, G9 protein, and/or A16 protein/G9 heterodimer of claim 26 and an adjuvant.

28. A method for manufacturing the vaccine composition of claim 1, comprising producing at least one poxvirus antigen proteins and attaching the at least one poxvirus antigen proteins to a nanoparticle.

29. A method for eliciting an immune response against an orthopoxvirus in a subject or for immunizing a subject against an orthopoxvirus, comprising administering an effective amount of the composition of claim 27 to the subject.
